(19) United States
(12) Patent Application Publication
(10) Pub. No.: US 2019/0365803 A1
(43) Pub. Date: Dec. 5, 2019

(54) NANOSTRAWS METHODS AND APPARATUSES

(71) Applicants: Nicholas A. MELOSH, Menlo Park, CA (US); Sergio LEAL-ORTIZ, Stanford, CA (US); Chris REHSE, Palo Alto, CA (US); Ryan SWOBODA, Orange Park, FL (US); Craig GARNER, Mountain View, CA (US)

(72) Inventors: Nicholas A. MELOSH, Menlo Park, CA (US); Sergio LEAL-ORTIZ, Stanford, CA (US); Chris REHSE, Palo Alto, CA (US); Ryan SWOBODA, Orange Park, FL (US); Craig GARNER, Mountain View, CA (US)

(21) Appl. No.: 16/431,696

(22) Filed: Jun. 4, 2019

Related U.S. Application Data
(60) Provisional application No. 62/680,421, filed on Jun. 4, 2018.

(51) Int. Cl. A61K 35/14; A61K 35/26; A61M 1/36; C12N 5/0783; C12N 15/89

(52) U.S. Cl. CPC A61K 35/14; A61K 35/26; C12N 2527/00; C12N 5/0636; C12N 15/89; A61M 1/3687

(57) ABSTRACT

Methods and apparatuses for extracting, modifying and returning cells to/from a patient to treat the patient. These apparatuses and methods may use nanostraws to deliver biologically relevant molecules such as DNA, RNA, proteins etc., into the cells rapidly and efficiently. The methods and apparatuses described herein are repeatedly capable of delivering biologically relevant cargo into cells with high cell viability, dosage control, unaffected proliferation or cellular development, and with high efficiency, including in a continuous or semi-continuous manner.

### CROSS REFERENCE TO RELATED APPLICATIONS

This patent application claims priority to U.S. Provisional Patent Application No. 62/680,421, filed on Jun. 4, 2018, which is herein incorporated by reference in its entirety. This patent application may also be related to U.S. patent application Ser. No. 16/038,062, filed on Jul. 17, 2018, and titled “APPARATUSES AND METHODS USING NANOSTRAWS TO DELIVER BIOLOGICALLY RELEVANT CARGO INTO NON-ADHERENT CELLS,” which claims priority to U.S. provisional patent application No. 62/534,511, filed on Jul. 19, 2017; each of these applications is herein incorporated by reference in its entirety.

### STATEMENT AS TO FEDERALLY SPONSORED RESEARCH

This invention was made with Government support under NSF award 1759075 (STTR II). The Government has certain rights in the invention.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Delivery of bio-molecules into cells is a crucial starting point for controlling cellular function, proliferation, differentiation, and apoptosis, among other things. Many bio-molecules will not spontaneously penetrate the cellular membrane, but rather need an external delivery method that will transport them into the intracellular space where they can perform certain tasks.

Current methods to deliver material (e.g., “cargo”) into cells, including non-adherent cells, have yielded low efficiencies, low cell viability, impacted cellular development or proliferation, been cargo specific, and/or was not suitable for sequential delivery. The lack of efficient delivery methods is currently a major hurdle that severely impacts research and commercial outcomes in fields spanning immunotherapy, gene therapy, agricultural development, transgenic animals, drug development, and much more.

Nanostraws have proven to be an efficient way to deliver biologically relevant cargo into cells. See, e.g., Vandersarl et al., Nano Letters. 12, 3881 (2012), and U.S. Pat. No. 9,266,725. U.S. Pat. No. 9,266,725 is hereby incorporated by reference in its entirety. However, nanostraw usage have so far been limited to certain cell types, and at limited efficiencies, thus limiting the use of nanostraws. Described herein are systems and methods for using nanostraws to deliver cargo for gene therapy or other processes.

### SUMMARY OF THE DISCLOSURE

Described herein are apparatuses for treating a patient by rapidly removing cells from the patient, transferring biologically relevant cargo into the cells using nanostraws, and returning the cells back to the patient. These methods and apparatuses for performing them may form a dialysis-like loop, and may be done rapidly (e.g., within hours or even minutes). In some variations the methods and/or apparatus may be configured to operate in a continuous or nearly-continuous fashion.

In particular the methods and apparatuses described herein may be done to a large number of cells without requiring the cells to rest and/or expand outside of the body; instead the cells may be quickly returned into the body. These methods and apparatuses may be configured to deliver gene therapies, small molecule therapies, or the like. In general, these methods and apparatuses may use nanostraws to deliver biologically relevant molecules (cargo) such as DNA, RNA, proteins etc., into cells (including non-adherent cells) such as immune cells, embryos, plant cells, bacteria, yeast etc. The methods described herein are repeatedly capable of delivering biologically relevant cargo into cells with high cell viability, dosage control, unaffected proliferation or cellular development, and with high efficiency. Among other uses, these new delivery methods will allow to scale pre-clinical cell reprogramming techniques to clinical applications.

Nanostraws are hollow metal-oxide nanotubes that extend from a surface. Nanostraws have been shown to be able to give direct access to the intracellular space of adherent cell types (cells that bind to a surface). The mechanism for gaining cellular access have been thought to be due to the cells gripping on to the surface and pulling themselves down onto the nanostraws by their own action, thereby stressing the cellular membrane just above the nanostraw tips. For that mechanism, it was hypothesized that only nanostraws of diameters smaller than about 50 nm and with very low areal density, would be able to give intracellular access for non-adherent cell types. Despite its industrial relevance, no data showing nanostraw-mediated intracellular access for non-adherent cells have been presented.

Herein described are apparatuses and methods that allow nanostraws (even of diameters larger than 50 nm) to gain intracellular access into non-adherent cells. The method may include placing cells in a suspension in a container with nanostraws and coupling it with an external force that will enable the cells to interact with the nanostraws, thereby providing a close contact in between the cells and the nanostraws. This external force can be exerted through a number of mechanisms, and is general in its application. Another version of the method is based on pressing nanostraws onto the cells. While in contact, biologically relevant cargo can be delivered into the cells through the nanostraws.

For example, described herein are methods of treating comprising: withdrawing fluid containing cells from a patient; applying force to drive the cells against nanostraws within a vessel of a treatment apparatus, wherein the nanostraws are on an inner surface of the vessel; transferring a material through the nanostraws into the cells; and returning the cells to the patient.

In general, the methods and apparatuses described herein use an external force in order to make a close contact between cells and the nanostraws. The external force can be a pressure, applying an electric and/or magnetic field, controlling osmotic and/or concentration gradients, use of surface interactions and/or species-species interactions, physical inducement such as centrifugal, flow, shear effects, and/or mechanical compression. Once the cells are in contact with the nanostraws, the cargo can be administered into the cells in any suitable way, including but not limited to diffusion through the nanostraws due to a concentration gradient, by an electric and/or magnetic field, by pressure, by osmotic gradient, by surface interactions and/or species-species interactions, by physical inducement such as centrifugal flow, and/or shear effects.

The examples provided herein for using an external force to enable delivery of molecules into non-adherent cells may be provided in a microfluidic embodiment.

In some variations, the method and apparatus may be adapted for continuous operation by withdrawing fluid (e.g., blood), and in some variations isolating, separating, and/or concentrating the cells to be treated (by delivering depot materials) within the apparatus, then exposing them to the nanostraws and applying force. In some variations, the apparatus may have a plurality of different regions (chambers, vessels, etc.) that operate sequentially, and/or serially; in some variations in parallel, to provide continuous treatment. Treated cells may be returned to the patient's body in all or some of the fluid removed.

In some variations, force may be applied by centrifugation. Centrifugation can be used to make cells in a suspension come in contact with nanostraws and allow for intracellular delivery of biologically relevant molecules into the intracellular space. For this method, centrifugation may be performed when the cells are in a container incorporating a nanostraw membrane. Once the container with the cells is centrifuged, the cells may be pelleted onto the nanostraws, allowing for intracellular delivery. Thus, the apparatus may include regions (e.g., cylindrical regions that are rotated and exposed to centrifugal force for brief periods in order to drive contact between the cells and the nanostraws. Centrifugal force may also be used to separate subtypes of cells to be treated, removed or the like.

Cargo can be delivered into the cells, either during the application of the force driving the cells onto the nanostraws, or just afterwards, when the cells are still in contact with the nanostraws. After a certain time, in the absence of additional force driving the cells onto the nanostraws, the cells can leave the nanostraws and go back into solution again, thereby setting an upper limit to how long one can wait after the centrifugation before the cargo has to be delivered. This upper time limit may be cell specific.

In any of the variations described herein, a binding agent may be used to bind the non-adherent tissue to the nanostraws. The binding agent may be cell-specific or general. For example, an antibody directed to an antigen present on the tissue (e.g., cell surface) may be bound to the nanostraws and/or the membrane from which the nanostraws extend. The binding agent (e.g., antibody) may help capture the tissue for contact with the nanostraws.

In any of the apparatuses and methods described herein, force can be applied to remove the cells from the nanostraws upon demand, including the use of centrifugal force, fluid flow, mechanical force (e.g., retracting the nanostraws, etc.), etc. In some variations one or more chemical and/or enzymatic agents may also or alternatively be used to remove the cells. For example, trypsin, collagenase, and/or other agents may be used to remove the tissue (e.g., cells) from the nanostraws. These agents may be particularly useful when a binding agent, such as an antibody, has been used to secure the tissue to the nanostraws.

For example, described herein are methods of delivering a biologically relevant cargo into non-adherent cells. These methods may include: applying a force to drive a suspension of cells into contact with a plurality of nanostraws, wherein the plurality of nanostraws extend through a substrate and a distance beyond the substrate that is between 2 nm and 50 μm, further wherein the plurality of nanostraws are hollow and have an inner diameter of between 5 nm-1500 nm; and driving the cargo from the nanostraws into an intracellular volume of the cells so that at least some (e.g., 20%, 25%, 30%, 35%, etc.) of the cells take up the biologically relevant cargo.

As used herein, the cells may take up the cargo by receiving the cargo into the intracellular space, including, but not limited to translocation of cargo into subregions of the intracellular space (e.g., the nucleus). Further, taking up cargo may include getting the cargo into the intracellular compartment above a predetermined threshold (e.g., a detectable level, including a level above background).

The force may be applied in any appropriate manner, including by moving the nanostraws to contact the cells, and/or centrifuging the suspension of cells to drive the cells into contact with the plurality of nanostraws. In some variations, the method includes incorporating magnetic particles on or in the cells, and applying force comprises applying a magnetic field to drive the suspension of cells into contact with the plurality of nanostraws.

In any of these variations, driving the cargo may include applying a pulsed electrical field. The cargo may be one or more of: nucleic acids or proteins.

Any of these methods may also include separating the cells from the nanostraws; for example, separating the cells from the nanostraws may comprise flowing a solution over the cells; and/or reversing the force applied to drive the suspension of cells into contact with the nanostraws.

In general, these methods may be performed on fluid containing the cells that is temporarily removed from the patient into an apparatus (e.g., a therapeutic apparatus); the cells may be treated, then returned to the body.

Within the apparatus, the nanostraws may be in fluidic communication with a fluidic passage connected to a reservoir of biologically relevant cargo. The suspension of cells may comprise plant cells. The substrate may be a porous structure. For example, a method of delivering a biologically relevant cargo into cells may include: applying a force to drive a suspension of cells into contact with a plurality of nanostraws, wherein the plurality of nanostraws extend through a porous structure and a distance beyond the porous structure that is between 2 nm and 50 μm, further wherein the plurality of nanostraws are hollow and have an inner diameter of between 5 nm-1500 nm; applying a pulsed electrical field to drive the cargo from the nanostraws into an intracellular volume of the cells so that at least 25% of the cells take up the biologically relevant cargo; and separating the cells from the nanostraws.

Also described herein are apparatuses for performing any of the methods described herein. For example, an apparatus for delivering a biologically relevant cargo into non-adherent cells may include: a first flow path, wherein the first flow path is configured to receive a fluid including cells (e.g., a suspension of cells); a deflectable substrate along a lateral side of the first flow path; a plurality of nanostraws extending through the deflectable substrate and into the first flow path, wherein the plurality of nanostraws extend from the flexible substrate, further wherein the plurality of nanostraws are hollow and have an inner diameter of between 5 nm-1500 nm; a reservoir in fluid communication with an inside of the plurality of nanostraws, the reservoir configured to hold the biologically relevant cargo; wherein the deflectable substrate is configured to deflect between a first position and a second position, further wherein the plurality of nanostraws extend further into the first flow path in the second position than in the first position to at least partially occlude the first flow path so that cell in the flow path communicate with one or more of the plurality of nanostraws. The apparatus may also include a fluid connection to the patient (e.g., a first fluid pathway from the patient to the apparatus and a second fluid pathway returning from the apparatus to the patient).

The apparatus may also include a pair of electrodes on opposite sides of the first flow path configured to apply an electrical field between the reservoir and the first flow path. These electrode may be configured (sized, positioned, located, etc.) to apply an electrical field between the reservoir and the first flow path when the deflectable substrate is in the second position. The apparatus reservoir may be configured as a second fluidic channel through which the biologically relevant cargo flows.

The first flow path may comprise an inlet at a first end and an outlet at a second end, and may extend between these two. The deflectable substrate may comprises a porous structure. The plurality of nanostraws may extend between 2 nm and 50 μm from the substrate. The deflectable substrate may be configured to transition from the first position to the second position based on the pressure in the reservoir. Any of these apparatuses may be configured (or may include) a cartridge.

Any of the methods described herein may include coupling the patient to the apparatus (e.g., a continuous-flow apparatus), further wherein returning the cells to the patient comprises returning the cells from the continuous-flow apparatus back into the patient.

Transferring material through the nanostraws to into the cells may comprise transferring material for a brief time period (e.g., less than about 30 min, less than about 25 min, less than about 20 minutes, less than about 15 minutes, less than about 12 minutes, less than about 10 minutes, less than about 7 minutes, less than about 5 minutes, less than about 1 minute, etc.). Thus, the cells may be coupled to the nanostraws for a very brief period of time. Alternatively, in some variations the cells may be maintained on the nanostraws for a longer period of time.

In general, the cells may be expanded (e.g., allowed to rest, grow, and in some cases divide) within the apparatus; alternatively, in some variations, the cells may be returned to the body relatively quickly, where they may be expand/grow within the patient's body.

As mentioned above, transferring the material into the cells may comprise transferring material from an inner diameter of the nanostraws wherein each nanostraw of the nanostraws are hollow and have an inner diameter of between 5 nm-1500 nm and each nanostraw extends between 2 nm and 50 μm from the inner surface.

Transferring material may further comprise applying a pulsed electrical field to drive the material from the nanostraws into an intracellular volume of the cells. Transferring the material may comprise transferring a biologically relevant cargo, as mentioned above (e.g., a polynucleotide such as a DNA, mRNA, plasmid, etc.).

The cells may be any appropriate cell type (e.g., blood cells).

As mentioned above, in some variations, the method may be very quick. For example, the steps of withdrawing, placing, applying and transferring are performed continuously over a period of less than 3 hours (e.g., less than 2 hours, less than 1 hour, etc.). The cells may be buffered within the apparatus using any appropriate buffer solution and the apparatus may regulate the solution.

For example, a method of treating a patient (e.g., a method of transforming a patient's cells using a dialysis-type cell transfection treatment) may include continuously: withdrawing fluid containing cells from a patient as part of a first circuit coupling the patient to a treatment apparatus; applying mechanical force to drive the cells against a plurality of nanostraws on an inner surface of the treatment apparatus; transferring a cargo material through the nanostraws into the cells comprising applying a pulsed electromagnetic field; and returning the cells to the patient though a second circuit portion coupling the patient to the treatment apparatus.

### BRIEF DESCRIPTION OF THE DRAWINGS

The novel features of the apparatuses and methods are set forth with particularity in the claims that follow. A better understanding of the features and advantages will be obtained by reference to the following detailed description that sets forth illustrative embodiments, in which the principles of the invention are utilized, and the accompanying drawings of which:

FIG. 1 is a schematic of a prototype apparatus for using nanostraws to deliver molecules into a plurality of cells

FIG. 2 illustrates examples of cargo that may be delivered by an apparatus such as the ones illustrated herein into a variety of cell types (shown on the right).

FIG. 3A (FIGS. 3A1-3A5) illustrates example of nanostraws and methods of fabricating them (e.g., by atomic layer deposition and plasma etching) that may be used as described herein.

FIG. 3B is a schematic of an apparatus including nanostraw for delivering material into a plurality of cells.

FIG. 3C illustrates an example of cells growing on surface including nanostraws (see profile on right of FIG. 3C); image on the left of FIG. 3C is a florescent stain showing cells.

FIG. 4 is a schematic of a method of treating a patient using an apparatus including nanostraws in a dialysis-like arrangement.

FIGS. 5A-5C show a side schematic view of an exemplary apparatus in which pressure (e.g., mechanical force) is used to press the cells onto the nanostraws by moving the nanostraws onto the cells. This variation may be referred to as a “French press” apparatus.

FIGS. 5D-5E show another variation of an exemplary apparatus similar to that shown schematically in FIGS. 5A-5C. In FIG. 5D, the bottom of the chamber may include a plurality of openings or pores through which fluid may be driven; these opening may be too small to allow the cells to allow passage of the cells, but the flow of fluid out of the chamber may drive the cells against the bottom allowing contact with the nanostraws, as shown in FIG. 5E, for delivery of cargo, as shown in FIG. 5F.

FIGS. 6A-6B show another example of a flow-through device in use. In FIG. 6A, a side view schematically shows the layout of the exemplary assembled device. Cells are injected through inlet 2, passing by underneath the nanostraws, and are collected at outlet 2. In FIG. 6B, the same side view shows the device during delivery of a cargo into cells flowing through the device. In FIG. 6B, the increased pressure in the cargo reservoir makes the nanostraw membrane bend, thereby coming in contact with the cells, allowing for intracellular access.

### DETAILED DESCRIPTION

Described herein are methods and apparatuses for delivering a load into cells using nanostraws. Non-limiting examples of nanostraws are provided below and in the figures.

For example, for nanostraws may have any appropriate dimensions, including diameters of between about 10-1000 nm and lengths of between about 0.1-25 μm.

For nanostraws of diameters 10-1000 nm and lengths of 0.1-25 μm, spacers in the range between 0 and up to 50 times the cell diameter can be used. For spacers larger than 10 times the cell diameter, the nanostraws will not be able to reach the cells for intracellular delivery. A spacer of about half the cell diameter may be suitable for delivery into embryos.

The nanostraws can be incorporated into a microfluidic setup in which biologically relevant cargo can be delivered into cells (including non-adherent cells). In such a setup, cells would be transported in microfluidic chambers and interact with nanostraws in one or several of the following ways: pressure, applying an electric and/or magnetic field, controlling osmotic and/or concentration gradients, use of surface interactions and/or species-species interactions, physical inducement such as centrifugal, flow, shear effects, and/or mechanical compression. Such a flow-through device would be able to greatly increase the number of cells that can be delivered into while at the same time being a closed system with minimal risk for contamination. In here, we give an example of one such embodiment in which a mechanical compression similar to the French press technique is used.

For example, nanostraws can have diameters ranging from 10 to 1000 nm and lengths ranging from 0.1-25 μm. Their density can be between about 104 to 1012 nanostraws/cm². U.S. Pat. No. 9,266,725 describes nanostraw geometry, methods of manufacturing nanostraws, and nanostraw compositions.

Cargo Solution: Buffer in which the cargo should be diluted can cover a wide range of salt concentrations. The solution in which the cargo is diluted can have an ion concentration ranging from 0 to 10 000 mmol/L. For larger cargo such as plasmid and proteins, we have seen the highest delivery efficiency using 0.1×PBS as the buffer to dilute the cargo in, regardless of type and strength of the applied external force, and regardless of cell type, see FIGS. 10a-10f for delivery efficiencies of Cas9 protein into Jurkat cells centrifuged onto 200×1200 nm nanostraws.

In some variations an electric field may also be applied. For nanostraws of diameters 10-1000 nm and lengths of 0.1-25 μm, a pulsed electric field can be applied over the nanostraws in order to aid the delivery of cargo into the cells. Pulse length from 1 μs to is [e.g., around 200 μs], pulse frequency from 0.01 Hz to 10 000 Hz [e.g., 40 Hz], duration of the applied pulses from 0.01 s to 10 h [e.g., around 40 s to 180 s]. The pulsed field can have any profile, including square. Static fields might also work for some niche applications. The electric field will enhance the transport of charged species through the nanostraws as well as to help permeabilize the cellular membrane just above the nanostraws. A pulse train of 15-25V, for 150-300 μs, at 30-50 Hz, for 80-120 s duration may be used in some variations. Higher voltage can be used.

Any of the nanostraw structures described herein may be part of an apparatus (e.g., device, system, etc.), including deices for transfecting or modifying cells. Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

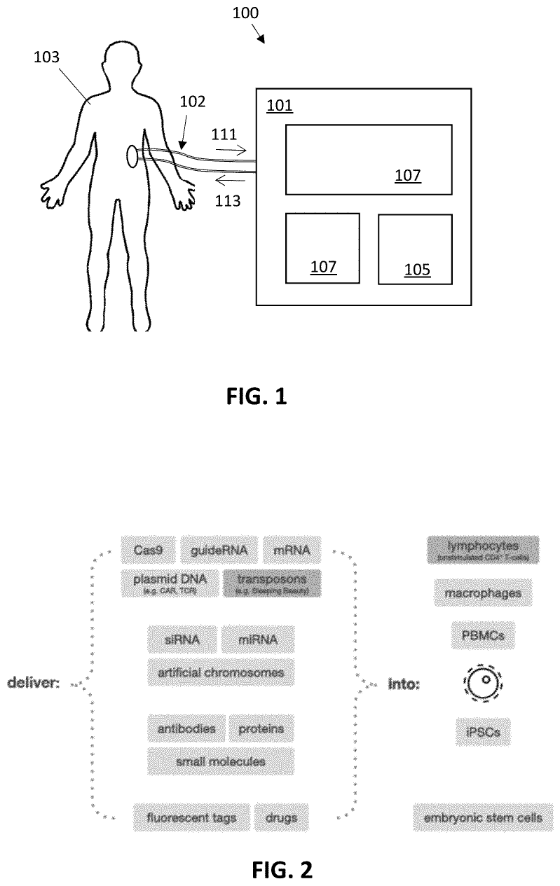

For example, FIG. illustrates one example of an apparatus that may be configured as a dialysis-like treatment apparatus for continuous or semi-continuous treatment of the a patient, removing cells (e.g., blood cells), treating cells and returning cells to the patient. Because these systems may rapidly deliver cargo into cells, they may be operated in a nearly continuous manner or continuous manner. In FIG. 1, the apparatus 100 is configured as a system that includes one or more patient connections 102 forming a circuit connecting patient fluid (with cells) to the rest of the apparatus 101. The circuit includes an inlet pathway portion 111 from the body to the apparatus and a return pathway portion 113 from the apparatus back to the body. Although separate lines are shown in FIG. 1 (which may return to the same body region or a different body region), in some variations a joint or combined line (having two portions) may be used, or multiple lines may be used.

The apparatus 101 may include a control 107 that may monitor operation of the apparatus, including fluid flow, temperature, force applied to the cells, energy (electromagnetic energy) applied, pH, etc. One or more sensors may be included (flow sensors, temperature sensors, pH sensors, etc.). The apparatus may include one or more chambers 107 for receiving cells and/or fluid, including nanostraws configured to deliver material to the cells. The system may also include one or more filters, concentrators, or the like 105 for separating our cells to be treated.

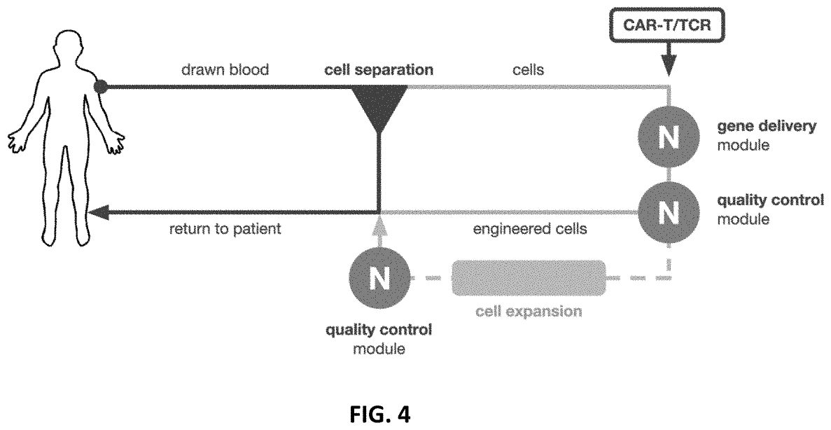

In use, the apparatus 100 may be connected to the patient 103 by coupling the inflow portion of the circuit 111 to the patient and the outflow portion of the circuit 113 to the patient and operating the apparatus. The apparatus may be primed first, e.g., so that the patient may receive flow in a continuous manner during treatment. The circuit may be attached to the patient at any appropriate body region (e.g., on a limb, trunk, etc.). FIG. 4 illustrates another schematic drawing of an apparatus (e.g., system), described in greater detail below.

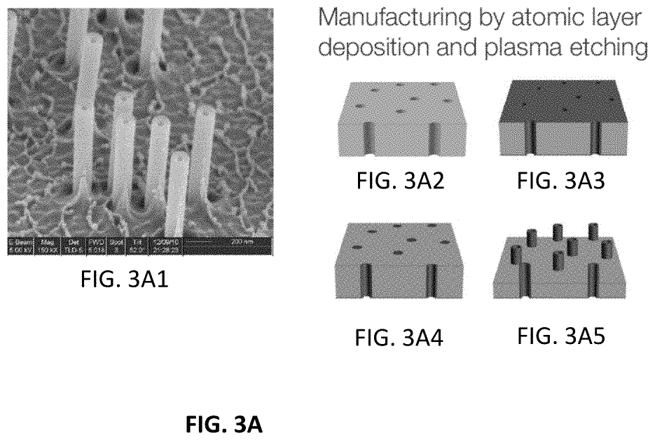

The apparatuses described herein may therefore be used to modify (e.g., “reprogram”) cells with minimal perturbation of the cells and may be used to deliver a variety of cargo materials, including combinations of materials. FIG. 2 schematically illustrates types of materials (e.g., nucleotides, antibodies, small molecules/drugs, etc.) that may be delivered using the nanostraws described herein, as well as the types of cells (e.g., blood cells, lymphocytes, macrophages, etc.). The cells may be isolated from the patient's fluid (e.g., blood) and/or may be added to the fluid removed from the patient following isolation separately.

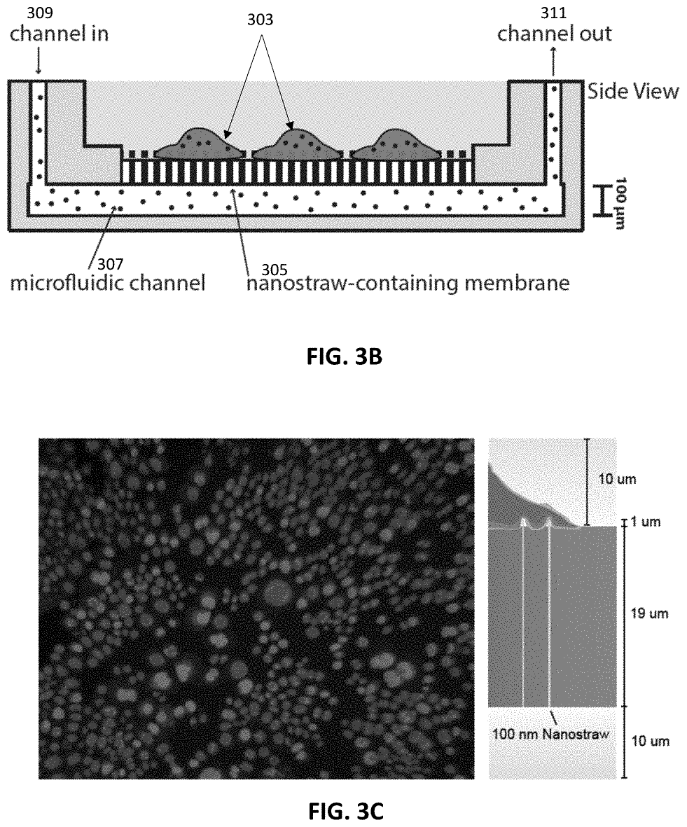

In general, nanostraws may be metal oxide nanotubes that include a central lumen through which material may be transferred. FIG. 3A illustrates one example of nanostraws (shown in the SEM of FIG. 3A1), and methods of fabricating them (showing steps 3A2-3A5). Nanostraws may be fabricated by, e.g., atomic layer deposition and plasma etching. Cells can be attached onto the nanostraws acutely (e.g., for a few minutes or hours), allowing temporary access without damaging the cells. For example, FIG. 3B schematically illustrates a portion of an apparatus including a chamber having a plurality of nanostraws. The cells 303 are at least temporarily attached to nanostraws extending through a base 305 (e.g., membrane, plate, etc.). A channel 307 is located behind the plate having the nanostraws and may carry fluid with one or more material (e.g., cargo) to be delivered into the cells. Fluid containing the cargo may be flowed into and out of the channel through one or more inlet 309 and outlet 311 ports. One or more pairs of electrodes (not shown) for generating an electromagnetic field may be included to apply electromagnetic energy to help drive the material into the cell from the nanostraws.

FIG. 3C illustrates one example of cells (shown on the left) transfected with a florescent marker by driving them onto nanostraws (shown schematically on the right); the florescent marker is driven into the cells as shown.

Another example of a system including nanostraws for delivering materials (e.g., cargo) into cells removed, transformed, and then returned to the patient's body. In FIG. 4 the apparatus includes a fluid circuit showing removal of blood from the patient and separation of the cells. The fluid separated from the cells may then be returned to the body via a return portion of the circuit. The target cells separated from the blood may then be driven against nanostraws including a cargo (e.g., a Chimeric Antibody Receptor Engineered T Cell/T-Cell receptors, CAR-T/TCR) that transforms or modifies the cells, e.g., T-cells, into therapeutic cells, such as improving the ability of T cell receptors to recognize and attack specific antigenic cell antigens by means of genetic modification. And therefore, they are collectively referred to as “T cell receptor redirection” technology. In this example, CAR-T directly changes one part of TCR into a specific antibody, allowing these modified T-cells to directly attack cancer cells under the guidance of antibodies. The T cell may invade cancerous tissue with its ability to identify cancer cells.

In FIG. 4, the apparatus may also include a quality control module, in which the cells are monitored and expression may be confirmed. The cells may optionally be allowed to expand or otherwise rest during this period. Once the quality control module confirms that the cells are ready (e.g., have been transformed), the cells may be returned into the body by inserting them back into the return path, as shown.

Thus, for example, T-cells may be driven onto the nanostraws as described herein, using a force (e.g., a centrifugation at a speed of about 200-2000 g for 2-20 min, e.g., approximately 750 g for 5 min). After the centrifugation, the cargo may be delivered before the cells go off in suspension again, generally before 30 min. Some cell types, such as large plant cells, can wait longer whereas some cell types, such as small T-cells, may be delivered to within 5 min.

In general, the buffer in which the cells are suspended during the centrifugation can be chosen to be any kind of liquid that will not lyse the cells. More viscous materials can be used to stabilize the cells on the nanostraws for a longer time and reduce the risk of cells going off into solution before the delivery has been performed.

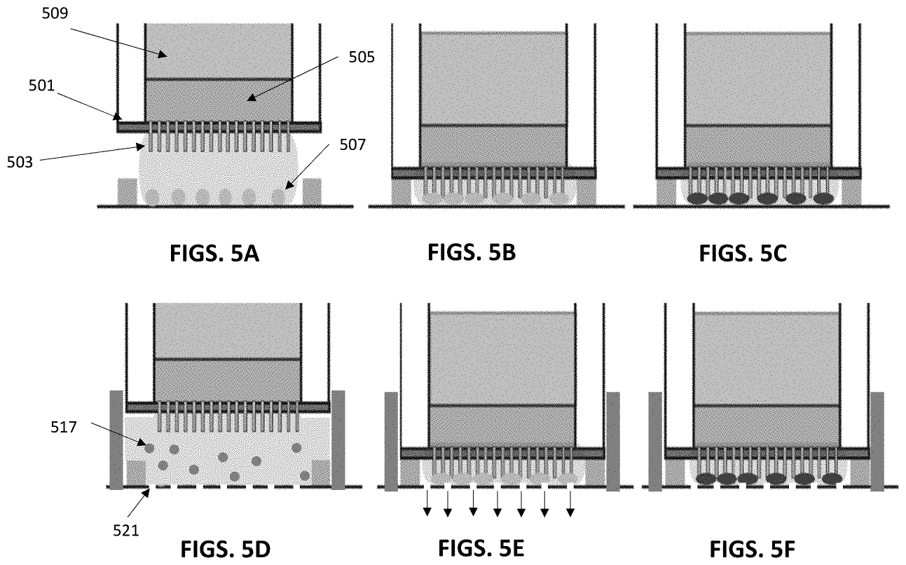

In some variations, mechanical force may be applied so that nanostraws can be pressed onto cells in order to gain intracellular access, the so called “French press” embodiment. In this example, illustrated in FIGS. 5A-5C and 5D-5E, a container incorporating a nanostraw membrane 501 in the bottom is filled with a cargo solution 505, FIG. 5A. Unlike the centrifugation-based method described above, since the nanostraws extend out from the container, facing down, cells are placed on a surface surrounded by a thin spacer whose height is chosen to match the height of the cells of interest. The nanostraw container 501 is placed on top of the cells such as to put the nanostraws and the cells in contact. If needed, extra pressure can be applied by adding another fluid on top of the cargo solution. Once a suitable pressure is applied, cargo can be delivered into the cells, including applying the transfer charge (e.g., applying a pulsed electric field).

When mechanical force is used to drive the cells into the nanostraws in the “French press” variation, cells may be dispersed on a flat surface surrounded by walls of a defined height. A container consisting of a porous membrane 501 incorporating nanostraws 503 may be extended downwards placed on top of the cells 507. The container may hold the cargo of interest 505 with the option of an extra weight e.g. oil 509 that can provide extra pressure. When pushed towards the cells (as shown in FIG. 5B), the cells will be squeezed, resulting in the nanostraws coming in close proximity to the cells. The nanostraws can then be used for delivery of a cargo into the cells, either by diffusion due to a concentration gradient or by adding external driving forces such as an electric field, centrifugation, liquid flow, etc.

Another variations is shown in FIGS. 5D-5F. In this example, as above, there is a receptacle for suspended, non-adherent cells 517 and the substrate with nanostraws may be moved into the receptacle; this may displace some of the fluid, which may pass through the base. The base in this example includes openings 521 (channels, pores, etc.) that may pass the fluid (as shown in FIG. 5E) when the substrate and nanostraws are lowered into the base, and may allow the substrate and nanostraws to drive the cells against the base so that they may be supported while the nanostraws contact them in order to pass cargo into the cells.

In variations in which the nanostraws are driven against the non-adherent cells, the pressure exerted by the nanostraws may be adjusted or controlled in order to control (e.g., tune) their penetration depth. Gene editing tools such as Crispr proteins can be delivered directly through the nanostraws allowing for quick and efficient genetic editing.

In some variations, a spacer may be used to adjust the depth of the nanostraws advanced into the cells. For nanostraws of diameters 10-1000 nm and lengths of 0.1-25 μm, spacers in the range between 0 and up to 50 times the cell diameter can be used. For spacers larger than 10 times the cell diameter, the nanostraws will not be able to reach the cells for intracellular delivery. A spacer of about half the cell diameter may be suitable for delivery into embryos. In general, nanostraws can be incorporated into a microfluidic setup in which biologically relevant cargo can be delivered into non-adherent cells. In such a setup, cells may be transported in microfluidic chambers and interact with nanostraws in one or several of the following ways: pressure, applying an electric and/or magnetic field, controlling osmotic and/or concentration gradients, use of surface interactions and/or species-species interactions, physical inducement such as centrifugal, flow, shear effects, and/or mechanical compression. Such a flow-through device would be able to greatly increase the number of cells that can be delivered into while at the same time being a closed system with minimal risk for contamination.

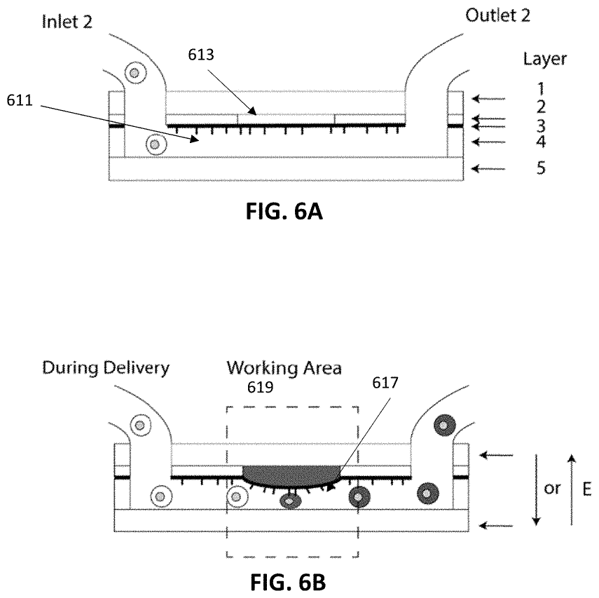

FIGS. 6A-6B show another example of a portion of a flow-through apparatus similar to that shown in FIG. 3B. In FIG. 6A, a side view schematically shows the layout of the exemplary assembled device. Cells are injected through inlet 2, passing by underneath the nanostraws, and are collected at outlet 2. In FIG. 6B, the same side view shows the device during delivery of a cargo into cells flowing through the device. In FIG. 6B, the increased pressure in the cargo reservoir makes the nanostraw membrane bend, thereby coming in contact with the cells, allowing for intracellular access. For enhanced delivery, an electric field, E, can be applied between the two ITO slides (over the nanostraw membrane and the cells, e.g., between the reservoir 605 and the channel 611 that the cells are flowing through). A working area 619 may include a deflectable substrate along a lateral side of the first flow path. The deflectable substrate may include a plurality of nanostraws 617 extending through the deflectable substrate and into the first flow path, wherein the plurality of nanostraws extend from the flexible substrate (e.g., the plurality of nanostraws may be hollow and have an inner diameter of between 5 nm-1500 nm). The apparatus also include a reservoir 613 in fluid communication with an inside of the plurality of nanostraws, the reservoir configured to hold the biologically relevant cargo. In this example, the deflectable substrate is configured to deflect between a first position (shown in FIG. 6A) and a second position (shown in FIG. 6B), further wherein the plurality of nanostraws extend further into the first flow path in the second position than in the first position to at least partially occlude the first flow path and secure non-adherent cell in the flow path against one or more of the plurality of nanostraws.

These apparatuses may be configured as open loop, closed loop or semi-open/semi-closed loop systems. A closed loop system may refer to the overall workflow, in which cells 1101 are injected into the device 1103, transformed, and returned in a sterile container 1105. This may reduce user handling, and enable higher numbers of cells to be transfected per unit time. In addition, it may allow the apparatus to be used clinically, where contamination concerns necessitate use of closed-loop devices. For example, the methods and apparatuses described herein may be used with CAR-T cell reprogramming at a local clinic, without need for technicians to manually oversee the transformation process (as schematically illustrated in FIG. 4, above).

In any of the variations described herein an electric field, and particularly a varying electric field, may be used to drive cargo into the cell and/or open the cell to the nanostraw. For example, for nanostraws of diameters 10-1000 nm and lengths of 0.1-50 μm, a pulsed electric field can be applied over the nanostraws in order to aid the delivery of cargo into the cells. Pulse length from 1 μs to 1 s, (e.g., between about 10 μs and 500 μs, between about 50 μs and about 500 μs, between about 150 μs and about 400 μs, about 200 μs, etc.) and pulse frequency from 0.01 Hz to 100,000 Hz (e.g., between about 0.1 Hz and about 50 kHz, between about 1 Hz and about 1 kHz, between about 10 Hz and about 100 Hz, about 10 Hz, about 20 Hz, about 30 Hz, about 40 Hz, etc.), a duration of the applied pulses from about 0.01 sec to 10 h (e.g., about 0.1 sec to about 1 hour, about 1 sec to about 5 minutes, about 10 sec to about 300 sec, between about 40 sec to about 180 sec, etc.). The pulsed field can have any profile, including square/rectangular, sinusoidal, triangular, etc. Static fields might also work for some applications. The electric field may enhance the transport of charged species through the nanostraws as well as to help permeabilize the cellular membrane just above the nanostraws. The efficiency of the electric field have so far found to be agnostic to the cell types; and exemplary parameters may include a pulse train of 15-25 V, for 150-300 μs, at 30-50 Hz, for 80-120 sec duration. In some variations, higher voltage can give higher transfection efficiency, but may lower the cell viability.

Any of the nanostraw structures described herein may be part of an apparatus (e.g., device, system, etc.), including deices for transfecting or modifying cells. Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating a patient, the method comprising:
withdrawing fluid containing cells from a patient;
applying force to drive the cells against nanostraws within a vessel of a treatment apparatus, wherein the nanostraws are on an inner surface of the vessel;
transferring a material through the nanostraws into the cells; and
returning the cells to the patient.

2. The method of claim 1, wherein applying the force comprises applying mechanical force.

3. The method of claim 2, wherein the mechanical force comprises moving the nanostraws to contact the cells.

4. The method of claim 2, wherein applying the force comprises centrifuging the cells to drive the cells into contact with the nanostraws.

5. The method of claim 1, further comprising releasing the force on the cells to release the cells.

6. The method of claim 1, wherein withdrawing fluid containing cells from the patient comprises coupling the patient to a continuous-flow apparatus, further wherein returning the cells to the patient comprises returning the cells from the continuous-flow apparatus back into the patient.

7. The method of claim 1, wherein transferring material through the nanostraws to into the cells comprises transferring material for less than 5 minutes.

8. The method of claim 1, wherein transferring materials comprises transferring for less than 1 minute.

9. The method of claim 1, further comprising expanding the cells.

10. The method of claim 1, wherein the transferring material comprises transferring material from an inner diameter of the nanostraws wherein each nanostraw of the nanostraws are hollow and have an inner diameter of between 5 nm-1500 nm and each nanostraw extends between 2 nm and 50 μm from the inner surface.

11. The method of claim 1, wherein transferring material further comprises applying a pulsed electrical field to drive the material from the nanostraws into an intracellular volume of the cells.

12. The method of claim 1, wherein transferring the material comprises transferring a biologically relevant cargo.

13. The method of claim 12, wherein the biologically relevant cargo comprises a polynucleotide.

14. The method of claim 1, wherein the cells comprise blood cells.

15. The method of claim 1, wherein the steps of withdrawing, placing, applying and transferring are performed continuously over a period of less than 3 hours.

16. A method of treating a patient, the method comprising continuously:
withdrawing fluid containing cells from a patient as part of a first circuit coupling the patient to a treatment apparatus;
applying mechanical force to drive the cells against a plurality of nanostraws on an inner surface of the treatment apparatus;
transferring a cargo material through the nanostraws into the cells comprising applying a pulsed electromagnetic field; and
returning the cells to the patient though a second circuit portion coupling the patient to the treatment apparatus.
